(19) United States
(12) Patent Application Publication
(10) Pub. No.: US 2024/0096480 A1
(43) Pub. Date: Mar. 21, 2024

(54) PROCESS MINING PATIENT WORKFLOWS FROM REAL-TIME LOCATION SYSTEM (RTLS) DATA

(71) Applicant: KONINKLIJKE PHILIPS N.V., EINDHOVEN (NL)

(72) Inventors: Pim Beekers, Eindhoven (NL); Supriyo Chatterjea, Eindhoven (NL); Alexander Sebastian Fumica, Eindhoven (NL); Ludovicus Marinus Geradus Maria Tolhuizen, Eindhoven (NL)

(21) Appl. No.: 18/039,738

(22) PCT Filed: Dec. 6, 2021
(86) PCT No.: PCT/EP2021/084298

§ 371 Date: Jun. 1, 2023

Related U.S. Application Data
(60) Provisional application No. 63/122,966, filed on Dec. 9, 2020.

(51) Int. Cl. G16H 40/20; G16H 40/67

(52) U.S. Cl. CPC G16H 40/20; G16H 40/67

(57) ABSTRACT

A system (10) for optimizing quality control of tracking data includes a real-time locating service (RTLS) (12) configured to perform tracking of persons or items in a building that are tagged with associated infrared (IR) or radio frequency (RF) tags (14) to generate candidate traces (40). At least one electronic processor (20) is programmed to: score the candidate traces including, for each candidate trace being scored, determine whether one or more location reports is missing from the candidate trace; and reduce the fitness score of the candidate trace based on a number of missing reports; detect deviations between the candidate traces and the intended workflow; and store the deviations in a database (23, 26).

### FIELD

The following relates generally to the tracking arts, medical procedure workflow arts, real-time location system (RTLS) arts, Petri Net process conformance checking workflow analysis arts, and related arts.

### BACKGROUND

Real-time location information allows hospital administrators, clinicians, and nurses to make decisions about patient treatment when and where needed. In a typical design, tracked people (e.g. patients, doctors, nurses, et cetera) and tracked objects (e.g. portable medical devices) are tagged with RFID or infrared (IR) tags, and stationary RF or IR tag readers or beacons can be strategically positioned throughout (the portion of) the hospital over which tracking is to be performed. In one design, the tags receive signals from nearby stationary beacons by which the location is determined at the tag, and the tag then re-transmits this location to an RTLS server. In another design, the tags transmit IR (or RF) signals that are received by stationary tag readers that convey the tag location to an RTLS server. In either case, the tracking data acquired by an RTLS is usually in the form of discrete position reports acquired at designated time intervals. In some RTLS, the time intervals between reports is variable, e.g. a longer time interval when the tracked person or object is stationary and shorter time intervals when the tracked person or object is moving as detected by a movement detector disposed in the tag. It is usually desirable to convert these discrete reports into tracks, e.g. a patient track delineating the path of a patient through the hospital. Such patient tracks can be analyzed individually to track deviations from a design-basis workflow, or can be aggregated (for example, the patient tracks of all patients following the design-basis workflow over a specified timeframe) to allow for statistical analysis of workflow deviations.

An illustrative example of a typical RTLS system is the CenTrak RTLS (available from CenTrak, Newtown, PA, USA). This RTLS comprises “monitors” that transmit unique infra-red (IR) signals; hence, the monitors serve as beacons. The system also comprises “stars” that serve as radio-receivers. A patient or staff member is provided with a wristband or other tag comprising an IR detector and a radio transmitter. The IR detector of the patient tag senses the IR signal of a nearby monitor (i.e., beacon) and sends an identifier of the monitor to the star via its radio. The star then communicates this location report to the RTLS server. The sending rate is adjustable and may depend on whether the tag moving or not. An advantage of using IR as the localization signal is that the infrared signal does not penetrate through walls and hence can offer room-level location granularity. On the other hand, the radio transmissions from the tags do penetrate walls—hence, a smaller number of stars (i.e. radio receivers) can be deployed compared with a larger number of IR beacons.

An RTLS system aimed at tracking patients introduces extra steps into the workflow of the staff, such as placing the wristband on the patient at the correct time and making sure it stays uncovered (clothing, bedsheets, et cetera may block the IR). These steps run in parallel to the actual care workflow and they have lower priority. This results in subpar RTLS data that makes its analysis difficult. Some resulting issues can include a staff member carrying multiple IR tags intended for patients, IR tags that are covered by blankets or clothing when attached to the patient (so that the IR signal is blocked), IR tags being attached to a patient after a workflow begins, IR tags being removed too late after completion of the workflow, among others. Such defects in the tracking data can make it difficult or impossible to process the tracking data to generate reliable patient (or object) traces.

The following discloses certain improvements to overcome these problems and others.

### SUMMARY

In one aspect, a system for optimizing quality control of tracking data includes a real-time locating service (RTLS) configured to perform tracking of persons or items in a building that are tagged with associated infrared (IR) or radio frequency (RF) tags to generate candidate traces. At least one electronic processor is programmed to: score the candidate traces including, for each candidate trace being scored, determine whether one or more location reports is missing from the candidate trace; and reduce the fitness score of the candidate trace based on a number of missing reports; detect deviations between the candidate traces and the intended workflow; and store the deviations in a database.

In another aspect, a system for optimizing quality control of tracking data includes a RTLS configured to perform tracking of persons or items in a building that are tagged with associated tags to generate an analysis trace. One or more localization stations are configured to determine a location of one or more of the associated tags based on communication between the associated tags and the one or more localization stations. At least one electronic processor is programmed to detect deviations between the analysis trace and an intended workflow using a Petri Net.

In another aspect, a system for optimizing quality control of tracking data includes a RTLS configured to perform tracking of persons or items in a building that are tagged with associated tags to generate candidate traces. At least one container is disposed in the building and includes a monitor for reading the associated contained in the container, the monitor blocking localization stations located outside of the container from reading the associated tags contained in the container. At least one electronic processor is programmed to: score the candidate traces including, for each candidate trace being scored: determine whether one or more location reports is missing from the candidate trace; add an interpolated report to replace any missing location reports in the candidate trace; assign a fitness score to the candidate trace indicative of how well the candidate trace matches an intended workflow; and reduce the fitness score of the candidate trace based on the number of added interpolated reports; select an analysis trace from the scored candidate traces based on the respective scores; detect deviations between the analysis trace and the intended workflow; and store the deviations in a database.

One advantage resides in addressing more flow issues more quickly and efficiently.

Another advantage resides in identifying deviations in a patient workflow from trace signals generated by IR tags.

Another advantage resides in identifying deviations in a patient workflow by comparing deviations in an RTLS data collection workflow and a candidate trace from a log of an IR tag.

Another advantage resides in resolving workflow issues from multiple IR tags, covered IR tags, and tags being attached ore removed at an incorrect time.

Another advantage resides in providing an apparatus for positively delineating the first and/or last report for a trace of a patient (or doctor, or object, et cetera) following a workflow.

Another advantage resides in providing an apparatus with tags in order to read an identification of a tag container.

Another advantage resides in providing for processing of tracking data to generate patient (or doctor, object, et cetera) trace that is robust against defects in the tracking data such as (but not limited to) missing location reports.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for optimizing quality control of patient traces in accordance with the present disclosure.

FIG. 2 shows exemplary flow chart operations of the system of FIG. 1.

FIGS. 3-5 show examples of guard structures used by the system of FIG. 1.

### DETAILED DESCRIPTION

The following relates to leveraging RTLS for a range of contemplated tasks. Some of these are retrospective, such as: monitoring patient stay length; providing workflow training data for artificial intelligence (AI) predictive algorithms; performing quality control as to compliance with intended workflows. Other contemplated tasks are more short-term or even real-time, such as providing feedback on workflow compliance in real time or per-shift or per day, e.g. as key performance indicators (KPIs) presented on a dashboard or in a daily report. The illustrative RTLS system uses infrared (IR) tags, which have an advantage of providing very high location certainty to a specific room (since the IR signal does not penetrate walls); however, the disclosed approaches can also be used with RTLS employing other types of tags, e.g. RF tags, or with a GPS-based RTLS.

In a typical workflow, a patient enters the hospital, an IR tag is taken from storage, its barcode is scanned into the patient's electronic medical record (EMR; sometimes referred to by other nomenclature such as an Electronic Health Record) to associate the tag to the patient, and the tag is secured to the patient, for example as a wristband. In some RTLS systems, such as the CenTrak system, the tag includes a bar code which can be scanned by a dedicated bar code scanner or a mobile device (i.e., a smartphone). The scanning devices stores the bar code of the tag in the EMR. The patient then goes through the workflow, for example receiving a medical imaging examination, going to surgery, being admitted to a hospital room (for an in-patient), or so forth. A disassociation step is performed at the end of the workflow to disassociate the tag from the patient. Similar tracking can be employed with mobile hospital assets such as portable ultrasound machines. For example, the IR tag may be associated to the ultrasound machine when it is checked out by a nurse or other medical professional, then used to perform ultrasound imaging of a patient, and finally disassociated from the ultrasound machine when it is checked back in. To disassociate the tag from the ultrasound machine, the tag is placed into a “dropbox” with a monitor contained therein.

Most of the previously listed contemplated tasks involve transforming the RTLS data (also referred to herein as tracking data) into patient traces or equipment traces. The raw RTLS data is in the form of location “reports” each including information such as a tag ID (which links to the patient due to the association step), timestamp, and location. The report rate may optionally be variable, e.g. ranging from every 5 minutes if the tag is stationary to every 3 seconds if the tag is moving. In principle, a patient trace is defined by the set of reports from when the tag is associated to the patient until disassociation. (While “patient trace” is used herein as an illustrative example, it will be appreciated that the trace can be for another type of person, e.g., a doctor or nurse, or for an asset as noted above, depending on the person or object to which the IR tag is attached. In addition, instead of a “patient” trace, the trace could be for a trace of a tag connected to a piece of medical equipment, i.e., an equipment trace).

However, there can be numerous difficulties with extracting patient traces. While the directional nature of IR tag detection is beneficial for localizing to a specific room, it also means that if the tag is obscured by clothing, intervening objects, or so forth then reports (which can contain location information) may be missed. Furthermore, some IR tags do not have on/off switches. This is a design choice motivated, for example, by the low power consumption of a typical IR tag (so that turning it off when not in use is not especially useful), cost considerations (a single hospital may employ many tags, and the useful life of a tag can be short due to wear-and-tear and sanitary considerations), and desire for ease of operation (requiring a nurse to turn a tag on when it is placed on a patient presents another possible “missed step” in the workflow; also, if a simple on/off switch is provided then it can be inadvertently or intentionally turned off by the patient). Because an IR tag without an on/off switch is “always on”, if the nurse takes the tag off the patient but then fails to disassociate it from the patient and carries the tag on the nurse's person, then the nurse's movements can be wrongly associated to the patient. In general, failure to timely associate/disassociate tags to/from patients creates the potential for missed patient traces or wrongly allocated “patient” traces.

In recognition of such problems, the following discloses several aspects or phases for improving quality control of the patient traces: (1) using containers or boxes to precisely delineate the start/end of a patient trace; (2) RTLS data cleanup and enrichment; (3) patient trace selection; and (4) patient trace analysis.

For aspect or phase (1), to address errors in associating/disassociating tags at the start/end of a workflow, a plurality of boxes are dispersed through a medical facility at which tags can be associated to a patient and disassociated from a patient. These boxes can be physical containers in which IR tags are stored for use or placed after use. In the CenTrak system, each box has an IR transmission device inside, transmitting a unique IR signal and acting as a beacon. The enclosure of the box block IR signals, so that tags inside the box read the unique IR signals from the IR transmission device inside the box, but no IR signals transmitted outside the box. If one or both of these reports is missing from a candidate patient trace, this (missing) information can be variously used to discard the trace, or the candidate trace can still be considered but the missing box report(s) can be flagged as workflow deviation(s).

In the cleanup/enrichment phase (2), the RTLS data are cleaned and enriched. Missing reports are addressed by adding interpolated reports to fill in the missing data. However, since such interpolations are estimates and may be in error, each interpolated report is identified as such by a flag or indicator, and optionally may be assigned an uncertainty, e.g. the uncertainty of each interpolated report may be increased if it has one or two temporally neighboring interpolated reports in a candidate patient trace.

In the patient trace selection phase (3), each candidate patient trace is assigned a fitness score that indicates how well the patient trace matches the intended workflow. A Petri Net is used for this purpose. A Petri Net is a known tool for analyzing temporal sequences; however, the following discloses using a Petri Net for the RTLS data analysis task. Optionally, guard structures may be added to the Petri Net. A guard structure limits the allowable trajectory exiting the guard structure based on the trajectory entering the guard structure, and thus adds some memory capability to the Petri Net. In another aspect of phase (3), patterns that are unrealistic for a patient trace may be used to discard certain reports. For example, if a patient tag visits many different locations this may be an indication that the tag is being carried by a nurse rather than attached to a patient, and these reports can be discarded.

The fitness score is computed for the entire candidate patient trace, and then this fitness score is preferably reduced based on the number of interpolated trace portions in the candidate patient trace. This latter reduction is a correction—since the interpolated reports are interpolated on the basis that the trace is assumed to be following the intended workflow, omitting this correction may result in the patient trace selection being biased toward selecting candidate patient traces with a large number of interpolated reports.

Finally, the patient trace analysis phase (4) selects trace(s) for analysis on the basis of the fitness scores, and then for each selected trace (also referred to herein as an analysis trace) the interpolated reports are removed and the (same) Petri Net is applied to the selected patient traces to detect deviations from the intended workflow. In some embodiments, the entire sequence of the patient trace can be analyzed, not only a subsequence of the patient trace with the best fit, for detection of deviations. Selection of the best fitting subsequence in these embodiments is used to determine the presence of deviations before or after the selected patient trace.

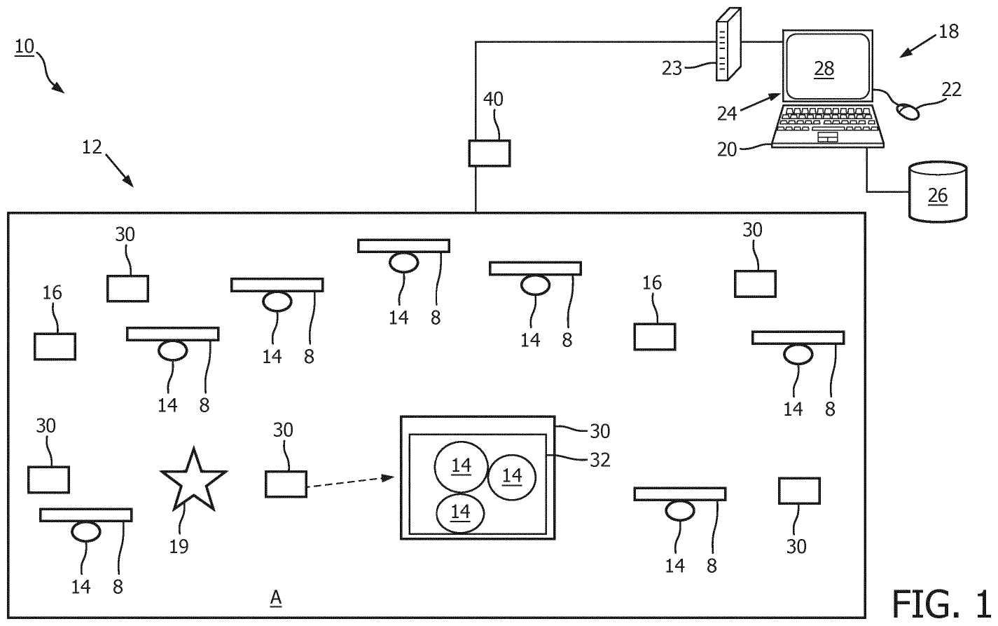

With reference to FIG. 1, an illustrative tracking system 10 for optimizing quality control of tracking data is shown. The tracking system 10 includes a real-time locating system (RTLS) 12 configured to perform tracking of persons (i.e., patients or medical personnel) or items (medical devices such as IV pumps, beds, equipment, and so forth) in a building A with tags 14 (e.g., radiofrequency identification (RFID) tags, or IR tags) and corresponding tag localization stations 16 (diagrammatically shown as rectangles in FIG. 1 which are placed at a priori-known fixed locations. In the Centrak system, for example, the tag localization stations 16 operate as beacons that transmit IR identifier signals which are detected by any tag 14 in proximity to the beacon. The tags 14 output location reports determined from the received beacon signals to radio receivers (called “stars” in the Centrak system) via radio transmissions to provide the location tracking. In other embodiments, the tag localization stations 16 may be tag readers that read an IR signal emitted by a tag 14 in proximity to the tag reader, so as to provide the location tracking. The localization stations 16 are distributed through a monitored area A and are configured to track locations of the tags 14 in the monitored area, either by operating as beacons as in the Centrak system, or by reading IR or RF signals emitted by the tags 14. For example, the tags 14 are attached or otherwise secured to one or more nodes 8 (e.g., a patient, a medical professional, a mobile object such as a piece of medical equipment, a zone in the building A, and the like). The tags 14 may be referred to by other terms, e.g. badges, tracking chips, et cetera—the term “tag” as used herein is intended to encompass such alternative nomenclatures. The localization stations 16 are distributed throughout the building A where persons or mobile objects to be tracked may traverse (e.g., in a patient room, in a hallway, at a workstation of a medical professional, and the like). In some examples, the building A can be a two-dimensional area (i.e., a single floor of a hospital) while in other examples, the monitored area can be a three-dimensional area (i.e., multiple floors of a hospital).

The localization stations 16 are configured to establish location data for proximate tags 14, thereby allowing the RTLS to track a corresponding node 8. In the simplest design, a localization station 16 may have an operational range, e.g. five meters, and any tag localized by that localization station 16 is known to be within a five-meter radius of the localization station. In other designs, two or three or more localization stations with overlapping operational ranges may operate in concert to locate a tag more precisely, e.g. using triangulation or the like. In yet other designs, a single localization station may provide directional information using a phased array transducer, a rotating transducer, or so forth. These are merely illustrative examples of RTLS ranging and angulating technologies, and more generally the RTLS 12 may use any suitable tracking technology to provide real time locational information for the tags present in the building A. In addition, the localization stations 16 are configured to transmit location beacons or receive tag identifying information from the corresponding tag 14 in order to enable the RTLS to determine the location of the particular tag 14 (and hence the corresponding node 8) being tracked.

The tag localization information may take various forms, e.g. in active tag designs the tag 14 includes an on-board battery-powered microprocessor or microcontroller and associated non-transitory memory (e.g. a FLASH, PROM, or other electronic memory chip) that stores a tag identifier number or the like which the tag 14 transmits to the localization station 16 which is in this embodiment a tag reader 16. In a passive tag design, radio frequency energy transmitted by the localization station 16 to the tag 14 powers the tag to drive it to transmit its tag identifier. In less sophisticated designs, each tag may transmit at a different frequency and the tag is identified by its response frequency. These are merely illustrative examples of tag identification technologies, and more generally the RTLS 12 may use any suitable tag identification technology to by which the tags 14 and localization stations 16 cooperatively generate real time location reports for the detected/tracked tags. Optionally, the RTLS 12 may be compliant with an industry-defined RTLS standard, e.g. ISO/IEC 24730-1 or a variant thereof.

In other embodiments, such as the CenTrak RTLS (available from CenTrak, Newtown, PA, USA), the tags 14 act as IR receivers and the localization stations 16 act as IR transmitters, i.e. IR beacons. The tags 14 are again configured to be worn by a hospital staff member or a patient or attached to a node 8. The tags 14 include an IR receiver and RF transceiver (not shown). The CenTrak system further includes a communication station 19 (shown in FIG. 1 as a star). The communication station 19 comprises a radio receiver that receives radio transmissions from the tags 14. These radio transmissions provide the tag locations based on the IR signals received from the localization stations 16 (which again in the CenTrak embodiment operate as IR beacons). Hence, each CenTrak tag 14 is configured to broadcast an IR signal with a unique ID representing a particular zone.

In either case (i.e., whether the localization stations 16 are tag readers or beacons), an identification (ID) of each localization station 16 is mapped to a particular zone in the building A. The tag locations are determined based on the communication with the localization station(s) 16 is reported to a server 23 of the RTLS. In the CenTrak system, the tag 14 itself is configured to report the sensed IR ID to the communication station 19 via an RF signal. In the alternative approach in which the localization stations 16 are tag readers, the stationary localization stations 16 are suitably connected with the server 23 via wired and/or wireless communication links so that the localization stations 16 (here tag readers) can report the locations of tags 14 based on the IR signals received from the tags. The server 23 is configured to map the IR ID of each tag 14 to the corresponding zone (which can be set by an installer of the RTLS 12). In other embodiments, the RTLS 12 can include any other location identification system, for example using RF, ultrasound, infrared or vision technology.

For conciseness, the term “tag” as used henceforth refers to the IR tags 14; however, the foregoing description is equally applicable to the RF tags. As previously noted, the difference in RF versus IR transmission modes has some impact on performance. For example, IR signals do not penetrate walls while RF signals generally do, so an IR tag can be localized to the specific room. By contrast, since RF signals can penetrate walls it is possible that a RTLS employing RF communication between the tags and the localization stations to perform the tag location could detect an RF tag in a different room. Multi-modality RTLS such as the CenTrak RTLS can combine these different signal types (e.g. IR and RF) to provide improved localization accuracy and performance.

The system 10 includes an electronic processing device 18, 23 such as workstation 18, a server computer 23, various combinations thereof, or more generally a computer. The server computer 23 may comprise a plurality of server computers, e.g. interconnected to form a server cluster, cloud computing resource, or so forth, to perform more complex computational tasks. The workstation 18 and/or server 23 includes typical components, such as an electronic processor 20 (e.g., a microprocessor), at least one user input device (e.g., a mouse, a keyboard, a trackball, a device with an embedded screen such as a tablet, a smartphone, a smartwatch, an alternate reality/virtual reality headset or goggles, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The system 10 also includes one or more containers 30 (i.e., a box) dispersed throughout the building A. For example, the containers 30 can be hung on walls, placed on shelves, be installed within walls (i.e., a drop-box) and so forth. The containers 30 are sized to hold tags 14 for pick-up by medical personnel for attachment to a node 8, or for drop-off by medical personnel after detachment from a node 8. The container 30 also includes a monitor 32 for reading associated tags 14 contained in the container. In addition, the monitor 32 is configured to block the tag localization stations 16 located outside of the container 30 from reading the associated IR or RF tags contained in the container. Advantageously, this prevents the “wrong” tags being read by the tag localization stations 16.

The non-transitory computer readable medium 28 stores instructions which are readable and executable by the at least one electronic processor 22 to perform

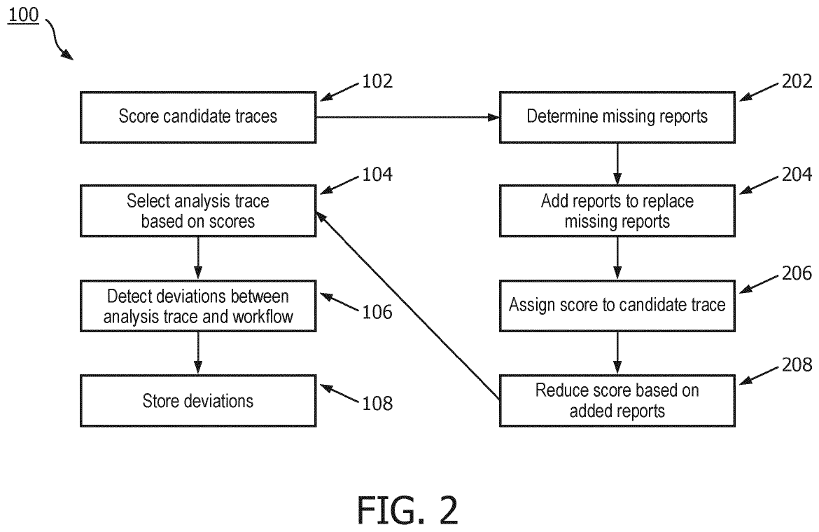

With reference to FIG. 2, and with continuing reference to FIG. 1, the computer 18, or the server computer 23 (or a combination of the two) is configured as described above to perform a method or process 100 for optimizing quality control of patient or equipment traces 40 generated by the tags 14, which are indicative of a trajectory over time of movement of the tags. Although described primarily in terms of patient tags 40, the following is also applicable for equipment or item traces 40. The non-transitory storage medium 26 stores instructions which are readable and executable by the at least one electronic processor 18 (or the server computer 23) to perform disclosed operations including performing the method or process 100. In some examples, the method 100 may be performed at least in part by cloud processing.

To begin the method 100, the patient traces 40 are generated by the tags 14 while the tags are picked up or dropped off at the various containers 30. The patient traces 40 can include location reports of the tags 14 throughout a time interval or trajectory during the treatment workflow. At an operation 102, the candidate traces 40 are scored. To do so, the operation 102 includes operations 202-208.

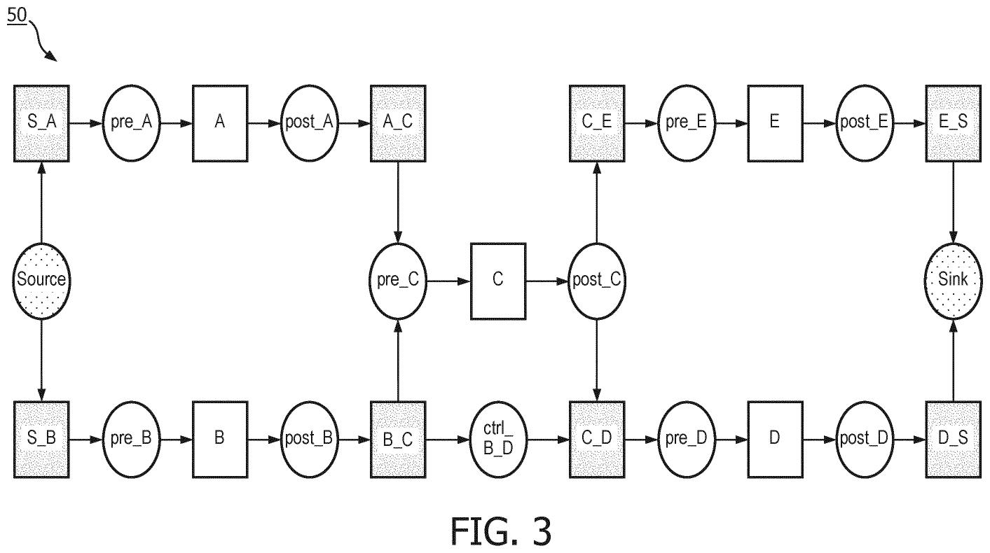

At an operation 202, one of the patient traces is selected as a candidate trace, which is analyzed to determine whether one or more locations reports are missing from the candidate trace. At an operation 204, interpolated reports are added to the candidate trace 40 to replace any missing location reports in the candidate trace. At an operation 206, a fitness score is assigned to the candidate trace indicative of how well the candidate trace matches an intended workflow (i.e., the workflow through which the tag 14 is being monitored). To assign the fitness score, a Petri Net process is applied to the candidate trace 40 to analyze temporal sequences of the candidate trace. In some examples, the applying to the Petri Net includes using one or more guard structures 50 (see, FIG. 3) of the Petri Net to provide the Petri Net with memory capability, as described in more detail below. Referring back to FIG. 2, at an operation 208, the fitness score of the candidate trace 40 is reduced based on the number of added interpolated scores. These operations 202-208 are repeated for each patient trace 40.

At an operation 104, an analysis trace is selected from the scored candidate traces based on the respective fitness scores. In some examples, the selection of the analysis trace from the scored candidate traces is further based on (1) whether a first report of the scored candidate trace is from the monitor 32 of the container 30 and (2) whether a last report of the scored candidate trace is from the monitor 32 of the container 30.

At an operation 106, deviations are detected between the analysis traces (i.e., the entire data stream or log) and the intended workflow. In some examples, the detection of the deviations between the analysis traces and the intended workflow includes detecting a first report of the analysis trace not being from the monitor 32 as a deviation. In other examples, the detection of the deviations includes detecting a last report of the analysis trace not being from the monitor 32 as a deviation.

In some embodiments, the deviations can be detected using the Petri Net operation (from the operation 206). In some examples, the operation 106 includes removing the interpolated reports from the analysis trace prior to applying the Petri Net to detect the deviations between the analysis trace and the intended workflow.

At an operation 108, the deviations are stored in a database, such as the non-transitory computer readable medium 26 of the workstation 18 or the server computer 23.

### Example

The following example describes the method 100 in more detail. The method 100 aims to identify errors that occur during the process of data collection for tags designated as patient tags. It is typically implemented as one or more software programs in the system 10. The system 10 analyzes cleaned and enriched RTLS location reports from the database, such as the non-transitory computer readable medium 26 of the workstation 18 or the server computer 23, and outputs the analysis results to the database.

A sequence of reports from the same tag is referred to as a “patient log”. The start and end of a patient log need not coincide with the start and end of care pathway or normative workflow. For example, hospital staff may walk around with a tag 14 before or after it serves as a patient tag. Therefore, one of the functionalities of the system 10 is the identification of patient traces 40, that is, determining when a tag starts and end having the role of a patient tag 14.

One aspect of the system 10 is the construction of a Petri net based on the “normative workflow” of a care-path in a hospital. The Petri net is used to identify patient traces from logs. This usage of the Petri net places strict requirements on the Petri net. Control structures are used in Petri nets in order to meet these requirements. These control structures serve a similar purpose as guards in so-called colored Petri nets (see, e.g., Wil M. P. van der Aalst, Christian Stahl and Michael Westergaard, “Strategies for modelling complex processes using coloured Petri nets”, in Transactions on Petri Nets and Other Models of Concurrency VII, Springer, 2013, pp. 6-55.).

The cleaned and enriched data can comprise reports that have been obtained by interpolation of raw RTLS patient traces 40 (e.g., corresponding to interpolation operation 204 of FIG. 2; if two consecutive reports of a tag correspond to different and non-adjacent locations then an interpolated report is added). As locations corresponding to a report obtained by interpolation have not actually been observed, there is some uncertainty about them. The data quality tool uses a criterion called “adjusted fitscore” for incorporating this uncertainty during candidate patient trace selection. This requires that during cleaning and enrichment of data, a reliability indicator is included in reports obtained by interpolation.

The Petri net is also used to analyze the identified workflow against the pre-determined workflow (operation 106 of FIG. 2), using standard conformance techniques from Petri nets in process analysis, like replay techniques.

In this analysis (that is, in performing the deviations detection analysis 106 of FIG. 2), reports obtained by interpolation are removed. Reports introduced because of missing report events are kept.

Prior to the trace selection operation 104, the system 10 preferably analyzes common errors in the collection of the data of a patient log. In case of a missing report event, a report is generated in which the monitor ID differs from all monitor IDs used in the RTLS system. This is done in order to detect these events during the analysis of identified traces.

In one embodiment, the analysis 100 provided by the system 10 is run on a regular, (i.e., daily), basis. Doing so requires some measures for analyzing in proper time windows. In another embodiment, a human user can prescribe a time interval from which data is analyzed.

To clean the candidate patient traces 40, data is read from the database which contains cleaned and enriched RTLS data. The cleaned and enriched data may comprise reports obtained by “interpolation” between consecutive reports of a tag that correspond to non-adjacent locations (corresponding to operations 202, 204 of FIG. 2). The choice for the locations obtained by interpolation may be pre-computed and stored for any pair of non-adjacent locations. It may be based on Dijkstra's shortest path algorithm, on a normative workflow, or on both. A reliability measure may be assigned to reports obtained by interpolation; an example of such a reliability measure is the number of shortest paths between the two non-adjacent locations. Each report is enriched with a Boolean indicating if the location has been inferred by interpolation or not, and with a reliability measure if the location had been inferred.

Errors may be made during data collection other than deviations in the patient trace (i.e., “general errors”). Tag IDs for which the log contains one or more type of general errors can be stored together with the type of errors. Standard type of errors can include: “Trace was too long”, “Missing report”, Missing Tag” (that is, the first report of the candidate trace is not from the monitor 32 inside the container 30), “Missing Container” (that is, the last report of the candidate trace is not from the monitor 32), and so forth. Tag logs containing general errors are not excluded from further analysis in order to select a patient trace.

Each tag 14 can send a report at least once every five minutes. Due to system failures and tags going out-of-range, no report may be received for a longer period. For each tag 14, it is checked when during the analysis interval no report was received for a longer time period than mis_rep_interval seconds. The default value, for example, can be mis_rep_interval=610 allows one report to be missed. Whenever the mis_rep_interval threshold between two consecutive reports of a tag is exceeded, the tag ID will be stored, as well as the location before and after the missing report. Keeping track of these locations might indicate where tags frequently go out-of-range. Moreover, for each missing report an extra location/row is added to the tag's log with a location ID not in use in the system. In this way, the Petri net replay operation will indicate missing reports as a deviation, both during patient trace selection and during analysis of selected patient traces.

For each tag 14, the number of locations can be counted, as this can indicate whether errors occurred during data collection. Indeed, pilot studies have shown that staff regularly walks around with patient tags, which causes noise in the data. Therefore, the user specifies a maximum number of locations a patient trace should have (max length traces; default=50). Tag IDs for which the log has more than max length traces locations will be stored; also, for these tags 14, a patient trace will be selected. The rationale for doing so is the use case where staff walks around with a patient tag before and/or after the tag is worn by a patient.

In order to help finding the start and end of a candidate patient trace 40, specific monitors may be installed and specific procedures may be followed. For example, patient tags 14 are kept in the container 30 at the location where patients enter the hospital. If a new patient arrives, a tag 14 is taken out of the container 30 and is placed in the vicinity of a localization station 16, e.g. placed under a desk. This way, the tag 14 is very likely to report the ID of this localization station 16, and traces of actual patient tags (that is, actually worn by patients) most likely start with this specific reader. Similarly, at locations where patients tend to leave the monitored area, another container 30 may be placed, in which tags 14 are collected and which has a monitor 32 inside. Another general error can be that a patient log does not comprise a report corresponding to the ID of the localization station 16 or the monitor 32.

For each tag 14, all potential sequences of consecutively visited locations (i.e. candidate traces) are obtained from the log. From all these candidate traces 40, the candidate trace that most likely corresponds to a true patient trace is selected. For doing so, all candidate traces are forwarded to a Petri net, which will output a fitness score which indicates how well the candidate trace matches the pre-defined workflow. Candidate traces 40 that overlap (have a common sequence of visited locations) with another candidate trace with a higher fitness score are excluded. Candidate traces with a fitness scores lower than a pre-defined level are excluded as well, in order to exclude candidate traces which are not likely to represent (complete) patient traces.

During enrichment of the data, missing locations were interpolated based on the last known and new locations. This is done to aid patient trace discovery as otherwise large parts of the workflow would be missing, which would interfere with selection of the patient trace. On the other hand, interpolating introduces uncertainty concerning the selection of the actual patient trace. To take this into account, an adjusted-fitness score may be computed which corrects for uncertainty due to interpolations. The adjusted-fitness score is calculated by multiplication of the local incremental fit score, which is the value the interpolation adds to the overall fitness score, by the local reliability score. The local reliability score represents the certainty of having interpolated correctly; and equals 1 divided by the number of shortest paths between the last known and the new location. The candidate trace 40 with the highest adjusted-fitness-score is considered the patient trace.

Expressed in formulas, the adjusted fitness score F_{adj}(T) of a trace T=(T₁, . . . , Tₙ) is computed as follows. k denotes the number of interpolations, and the start and end index of interpolation i be denoted by sᵢ and eᵢ, respectively. For 1≤i≤k, the local increment Δᵢ is defined as Δᵢ=F(T)−F(T₁, . . . , Tₛ_{ᵢ}₋₁, tₑ_{ᵢ}₊₁, Tₑ_{ᵢ}₊₁, . . . , Tₙ). Here F denote the fitness score. The local increment can be seen as the contribution of the interpolation to the overall fit score. The adjusted fit score is computed as

$${F}_{adj} ( T ) = F ( T ) - {\sum }_{1 \leq i \leq k} {Δ}_{i} + {\sum }_{1 \leq i \leq k} \frac{{Δ}_{i}}{{N}_{i}} ,$$

where Nᵢ denotes the number of paths of minimum length between Tₛ_{ᵢ}₋₁ and Tₑ_{ᵢ}₊₁, the locations just before and after interpolation i.

If the above specific monitors and procedures are in place, then the start and end locations of the selected patient trace 40 correspond to these specific monitors, and no Petri net is required for patient trace 40 selection if both a start and an end monitor appear in a patient log. When both monitors are missing, the process will be initiated. Whenever only the start or end monitor is detected, locations before or after are excluded, respectively. Additionally, the process is initiated on the remainder of locations in the log, where either the start or end location of the trace 40 is fixed.

The traces 40 obtained after patient trace selection are investigated for potential deviations. Reports obtained by interpolations are removed; and reports introduced due to a missing report event are kept. The remaining trace 40, consisting of the original patient trace and reports introduced due to a missing report event, can be analyzed by applying to the Petri net the alignments technique originating from the field of process mining to do conformance checking. This technique compares the patient trace to the model and shows where the patient trace deviates from the model. More specifically, it shows where a location is expected but not observed, and also where locations have been observed but were not expected according to the workflow. These deviations will be stored in the database 18, 23 along with other identified errors, information regarding patient trace selection, the selected patient traces, and the logs of all tags 14.

The system 10 uses this Petri for selecting a patient trace 40 and identifying deviations in selected traces. Petri nets are a popular tool in process mining and analysis (see, e.g., Wil M. P. van der Aalst, “Process Mining”, Springer, 2011).

A Petri net consists of places which can hold tokens, transitions which consume and produce tokens, and arcs that connect places and transitions. Additionally, a Petri net can contain hidden transitions. Hidden transitions do not correspond to actual parts of the workflow but are necessary for routing purposes; their executions are never recorded in event logs. Transitions in the Petri net correspond to locations in a workflow. Real-life transitions between locations in the workflow are referred to as cross-overs, in order to avoid confusion with transitions in a Petri net. Petri net transitions representing locations will be referred to as location transitions.

Another type of transition represents a cross-over and is referred to as cross-over transition. Cross-over transitions are used as they provide additional information on the direction of the corresponding transition between locations, whereas location transitions only provide information on the occurrence of a location. In order to connect location transitions to cross-over transitions, pre- and post-places are used.

In a first step, a simplistic Petri net for the normative workflow is generated. The user manually generates a list of all valid crossovers (that is, all transitions from one location to another one that occur in at least instantiation of the normative workflow). A software program extracts all unique locations from this list and adds them to the Petri net as transitions. Pre- and post-places are added to the net and connected to the corresponding location transitions. Using the list of valid crossovers, cross-over transitions are added to the net and connected to the pre- and post-places of corresponding locations transitions.

The simplistic Petri net structure described above is underfitting as it allows for invalid workflows due to intersections between valid workflows. For example, in workflow 1, a patient can move from location A to location E through C. In workflow 2, a patient can move from location B to D through C. The Petri net constructed so far also allows a patient to move from location A to location D through C. To prevent that such trajectories not conforming to the normative workflow go unnoticed, information on enabled cross-over transitions (independent) is used to determine what other cross-over transitions (dependent) can be enabled later in the workflow. This information can be stored in so called ‘control places’ by inserting a token in it by the independent cross-over transition. In this way, control places memorize past transitions. The dependent cross-over transition requires input from the control position in order to be activated. Therefore, these control places act as a guard by using memory to prevent incorrect flows in the Petri net. Guards are also used in colored Petri net having the same functionality.

The input given by the user for creation of a Petri net are: List of valid crossovers: [Loc A, Loc B]; List of dependencies: [First location of dependent crossover, second location of dependent crossover, first location of independent crossover, second location of independent crossover, Type of the dependency(optional/mandatory)]; List of start locations; and List of end location

In the first step, a simplistic Petri net is generated, as described above. Next, guards 50 are added to ensure that the Petri net only allows for valid paths, using the list of dependencies specified by the user. For each of these dependencies the guards are added to the net. The guard type, indicating if the dependent crossover transition is optional or mandatory, depends on what the user specified.

FIG. 3 shows an example of a guard structure 50. The ovals indicate positions (in which the green oval is the source position and the red oval is the sink position), the squares are transitions (the black squares indicating hidden transitions) and the arcs indicate connections between transitions. The independent cross-over transition ‘B_C’ feeds into the pre-position of location transition C and into the control position (ctrl_B_D in the figure below). The dependent cross-over transition ‘C_D’, requires the token from this control position ‘ctrl_B_D’. Thus, the independent transition ‘B_C’ enables the dependent transition ‘C_D’ later in the Petri net. FIG. 3 illustrates that the transition from C to D is only allowed if there was a transition from B to C. An extra feature of this structure is that the independent cross-over transition forces the dependent cross-over transition to be enabled, as otherwise one token would remain in the control position. Therefore, these structures are referred to as mandatory dependencies.

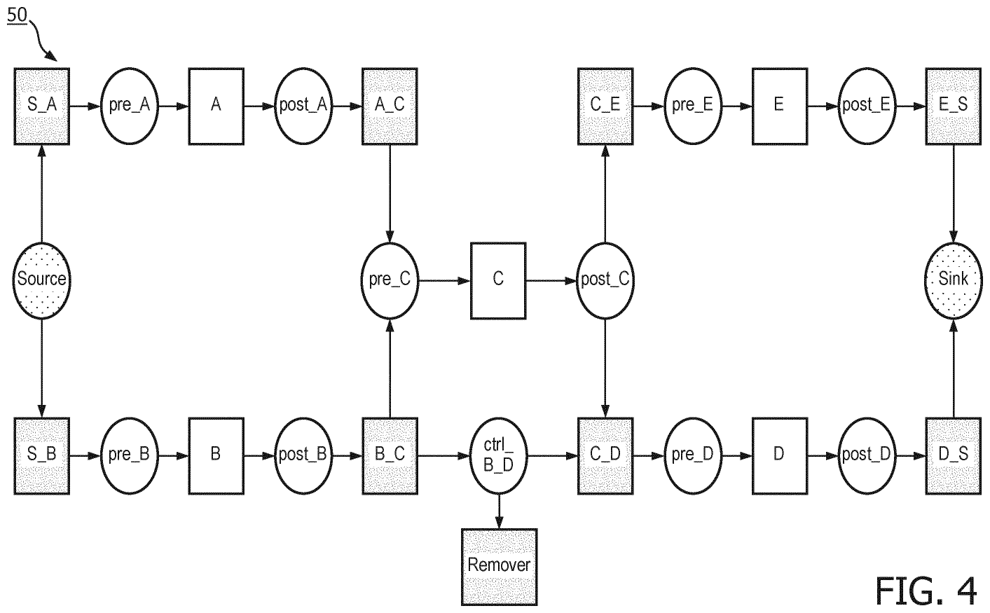

If it is not desirable to enforce a dependent cross-over transition, an extra hidden transition can be added, which consumes the token generated in the control position without outputting a token. This extra hidden transition is referred to as a ‘REMOVER’. An example is shown in FIG. 4. In this figure, the transition ‘C_D can only be enabled if the transition ‘B_C’ was enabled, but enabling of transition ‘C_D’ is not mandatory because the token that would remain in the prior example can now be removed by the “REMOVER” transition.

A cross-over transition T can be dependent on multiple cross-over transitions. It is a mandatory cross-over transition for a non-empty set M of cross-over transitions, and an optional cross-over transition for a set non-empty set O of cross-over transitions. A control structure is employed to deal with this situation. Two control places, ctrl_T_M and ctrl_T_O, are created. All cross-over transitions from M and O are connected with ctrl_T_M and ctrl_T_O, respectively. The control place ctrl_T_M and ctrl_T_O thus can collect tokens from the cross-over transitions from M and O, respectively. As ctrl_T_M and ctrl_T_O have different functionality, they cannot be combined into one control place. To resolve this, hidden transitions referred to as ‘doublers’ place tokens in both places, I.e., crtl_T_M and ctrl_T_O for ensuring that the crossover transition T can be enabled.

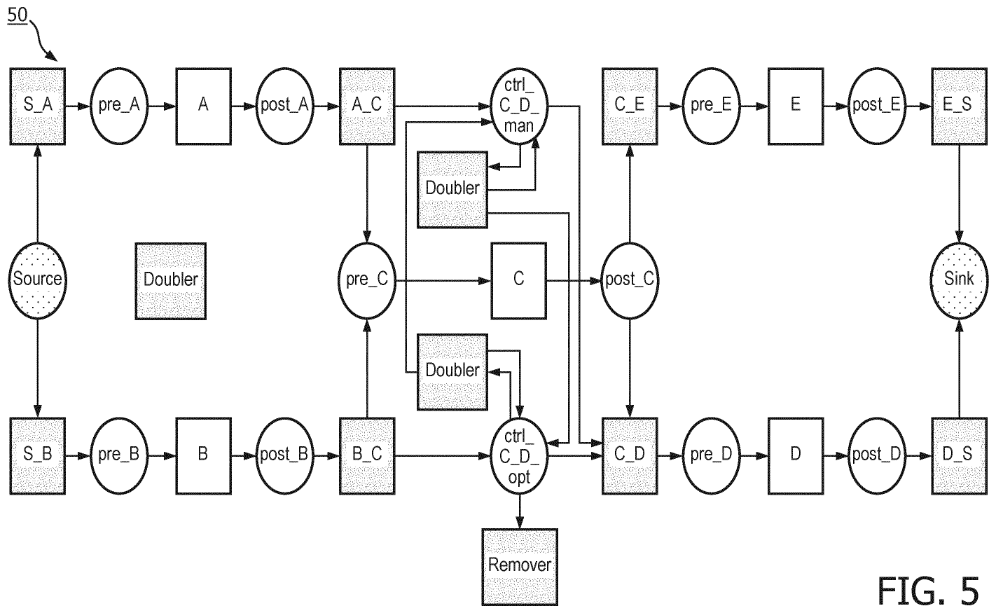

In FIG. 5, an example is shown. In this example, crossover transition ‘C_D’ is dependent on crossover transition ‘A_C’ and ‘B_C’ in which the types of dependencies are mandatory and optional, respectively. When B_C is enabled, it will output a token in ‘pre_C’ and in ‘ctrl_C_D_opt’. At this point it is not yet possible to enable ‘C_D’ as there is no token in ‘ctrl_C_D_man’. One of the ‘doubler’ transitions replicates a token in ‘crtl_C_D_opt’ and places a token in ‘ctrl_C_D_man’, in this way C_D can be enabled.

In some scenarios, a cross-over transitions may only be enabled at most a particular number of times, for example, at most k times. In this case, a special type of control-positions referred to as a budget-position, is added as input to the cross-over transition. The budget-position has no input; upon initialization, k tokens are added to this control-position. Finally, a REMOVER is added to this budget-position. A REMOVER is not required if a location must be visited exactly k times. When a cluster of cross-over transitions may only be visited k times, the budget-position is connected to all cross-over transitions.

The current implementation of this feature significantly increases the runtime of Petri net evaluations methods such as alignments and token replay. The feature therefore currently is not part of the Petri net generation module; it will be added once the runtime issue has been resolved.

The system 10 is configured to process a triple (d,i,t) of dependent crossover transition, independent crossover transition, and type t (that is, t is “optional” or “mandatory”), as specified by the user. First it is checked if the Petri net contains a controller c of type t with an outgoing arc to d. If so, then an outgoing arc from I to c is added to the Petri net. If not, the following steps are taken. First, a control position c′ is added to the Petri net, as well as an outgoing arc from c′ to d, and an outgoing arc from I to c. If t is “optional”, a (hidden) REMOVER transition is added to the Petri net, with an incoming arc from c′. Finally, if the Petri net already has a controller c″ of type different from t and with an outgoing arc to d, then two doublers are added to the Petri net. For both doublers, outgoing arcs to both c′ and c″ are added. An outgoing arc from c′ to one of the doublers is added, as well as an outgoing arc from c″ to the other doubler.

In sum, several aspects or phases for improving quality control of the patient traces are disclosed, including: (1) using containers or boxes to precisely delineate the start/end of a patient trace; (2) RTLS data cleanup and enrichment; (3) patient trace selection; and (4) patient trace analysis. In general, each of these various aspects or phases may be employed alone, or in various combinations. For example, the disclosed containers or boxes may be deployed in conjunction with the analysis aspects (2)-(4), or may be deployed in conjunction with some other type of analysis.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system for optimizing quality control of tracking data, the system comprising:
a real-time locating service configured to perform tracking of persons or items in a building that are tagged with associated infrared (IR) or radio frequency (RF) tags to generate candidate traces; and
at least one electronic processor programmed to:
score the candidate traces including, for each candidate trace being scored:
determine whether one or more location reports is missing from the candidate trace; and
reduce the fitness score of the candidate trace based on a number of missing reports;
detect deviations between the candidate traces and the intended workflow; and
store the deviations in a database.

2. The system of claim 1, wherein the at least one electronic processor is further programmed to:
add an interpolated report to replace any missing location reports in the candidate trace;
assign a fitness score to the candidate trace indicative of how well the candidate trace matches an intended workflow; and
reduce the fitness score of the candidate trace based on the number of added interpolated reports.

3. The system of either one of claim 1, wherein the at least one electronic processor is further programmed to assign the fitness score by:
applying a Petri Net process to the candidate trace to analyze temporal sequences of the candidate trace.

4. The system of claim 3, wherein the applying of the Petri Net includes:
using one or more guard structures of the Petri Net to provide the Petri Net with memory capability.

5. The system of either one of claim 3, wherein the applying of the Petri Net includes:
removing the interpolated reports from the analysis trace prior to applying the Petri Net to detect the deviations between the analysis trace and the intended workflow.

6. The system of claim 3, wherein the at least one electronic processor is further programmed to:
detect the deviations using the Petri Net.

7. The system of claim 1, further including:
one or more localization stations configured to determine a location of one or more of the associated tags based on communication between the associated tags and the one or more localization stations;
at least one container disposed in the building, the container including a monitor for reading associated IR or RF tags contained in the container, the monitor blocking the localization stations located outside of the container from reading the associated IR or RF tags contained in the container.

8. The system of claim 7, wherein the at least one electronic processor is further programmed to:
select an analysis trace from the scored candidate traces based on the respective scores, the selection of the analysis trace from the scored candidate traces being based on (1) whether a first report of the scored candidate trace is from the monitor or (2) whether a last report of the scored candidate trace is from the monitor.

9. The system of claim 7, wherein the detection of deviations between the candidate traces and the intended workflow includes detecting a first report of the analysis trace not being from the monitor as a deviation.

10. The system of claim 7, wherein the detection of deviations between the candidate traces and the intended workflow includes detecting a last report of the candidate traces not being from the monitor as a deviation.

11. (canceled)

12. (canceled)

13. (canceled)

14. (canceled)

15. (canceled)

16. The system of claim 1, the system comprising:
at least one container my disposed in the building, the container including a monitor for reading the associated contained in the container, the monitor blocking localization stations located outside of the container from reading the associated tags contained in the container.

17. (canceled)

18. (canceled)

19. The system of claim 16, wherein the selection of the analysis trace from the scored candidate traces is further based on (1) whether a first report of the scored candidate trace is from the monitor and (2) whether a last report of the scored candidate trace is from the monitor.

20. The system of claim 19, wherein the detection of deviations between the analysis trace and the intended workflow includes at least one of:
detecting a first report of the analysis trace not being from the monitor as a deviation; and
detecting a last report of the analysis trace not being from the monitor as a deviation.
